(19) United States
(12) Patent Application Publication
(10) Pub. No.: US 2015/0216700 A1
(43) Pub. Date: Aug. 6, 2015

(54) Shoulder support sleeve and vest

(71) Applicant: Jessica Paparella, Deer Park, NY (US)

(72) Inventor: Jessica Paparella, Deer Park, NY (US)

(21) Appl. No.: 13/999,268

(22) Filed: Feb. 5, 2014

(51) Int. Cl. A61F 5/01

(52) U.S. Cl. CPC A61F 5/012; A61F 5/013; A61F 2250/0003

(57) ABSTRACT

A shoulder support sleeve and vest having a vest section oriented on the torso of a patient, and a sleeve section integrated with the vest section, which is placed on an affected shoulder and arm portion of the patient. An inflatable bladder is attached to a back of the sleeve section and the vest section. An air pump is attached to the inflatable bladder, and is positioned to be accessible by the patient's non-affected arm/hand. The patient uses the air pump to inflate the inflatable bladder device to support the affected shoulder of the patient. The inflatable bladder can be removably attached to the sleeve and vest sections.

### CROSS-REFERENCE TO RELATED APPLICATIONS

This application claims priority to U.S. Provisional Patent Application 61/850,067, filed Feb. 8, 2013, entitled “Sleeping Sleeve: A comfortable nights' sleep for the ‘Sleeping Shoulder’”, to Jessica Paparella, and U.S. Provisional Patent Application 61/964,086, filed Dec. 17, 2013, entitled “The Shoulder Sleeve: A comfortable nights' sleep for the ‘Sleeping Shoulder, an updated version” to Jessica Paparella.

### FIELD OF THE INVENTION

This invention relates to an apparatus for use in rehabilitation or post-surgical operations relating to the shoulder, and more specifically, to a shoulder support sleeve and vest for use when a patient is lying flat and/or as a supportive brace during activities of daily living.

### BACKGROUND OF THE INVENTION

People with shoulder pathology (e.g., adhesive capsulitis, humeral fracture, rotator cuff repair, labral repair, and other post-surgical cases) have complaints of inability to sleep while laying flat in bed because of several theories, including: (i) the humeral head of the shoulder drops back into the glenoid (shoulder socket), which causes a stretch to the anterior structures of the shoulder; (ii) the superior elevation of the scapula (rising of the ‘shoulder blade’) while laying supine (flat); and/or (iii) the humerus (upper arm bone) extends, or drops back, past the midline of the body. Initial recovery generally requires immobilization, in sling, for up to 6 weeks as determined by a doctor.

While recuperating, patients seeking to lay supine (flat) generally prop a pillow under their shoulder for support, but typically the pillow slips out or is incorrectly placed, causing even greater discomfort. In such cases, patients typically resort to sleeping in a recliner/sitting to help alleviate pain, which is caused by one or more of the three theories mentioned above.

What is needed therefore, is a shoulder support sleeve and vest that is designed such that even when a patient is supine, the shoulder is placed in an anatomical position that mimics the shoulder position of the patient's arm resting at side, with slight shoulder abduction (movement of the arm to the side of the body) and flexion (movement of the arm to the front of the body).

The shoulder support of the present invention is designed to maintain typical anatomical position of the humeral head in the glenoid (center the ball in the socket), inhibit superior elevation of the scapula, and/or position the humerus in the midline of the body. The shoulder support of the present invention is adjustable, allowing health care practitioners to adjust the brace to meet the individual needs and anatomy of each patient.

### SUMMARY OF THE INVENTION

It is therefore an object of the present invention to provide a shoulder support sleeve and vest, having a vest section oriented on the torso of a patient, and a sleeve section integrated with the vest section, which is placed on an affected shoulder and arm portion of the patient. An inflatable bladder is attached to a back of the sleeve section and the vest section. An air pump is attached to the inflatable bladder, and is positioned to be accessible by the patient's unaffected arm/hand. The patient uses the air pump to inflate the inflatable bladder device to support the affected shoulder of the patient. The inflatable balder can be removably attached to the sleeve and vest sections.

The vest and sleeve sections can also include a strap attached to a front portion of the vest, which extends diagonally to the back of the affected shoulder. In addition, an outer pouch can be attached to the lower front portion of the vest to support the arm, especially while lying flat.

The invention can also include a foam material placed on a side of the vest adjacent to the patient, and the foam material may be removably attached.

It is another object of the present invention to provide a method of supporting a shoulder while lying supine (flat), the method including providing a vest section oriented on the torso of a patient, and a sleeve section integrated with the vest section, and placed on an affected shoulder and arm portion of the patient. An inflatable bladder is provided that is attached to the sleeve section and the vest section. An attached air pump is connected to the inflatable bladder, and is accessible by the patient's unaffected arm and hand, and is used to inflate the inflatable bladder to support the affected shoulder of the patient while lying flat.

### BRIEF DESCRIPTION OF THE DRAWINGS

FIG. 1 is a front view of the shoulder sleeve support and vest of the present invention; and

FIG. 2 is a back view of the shoulder sleeve support and vest of the present invention.

### DETAILED DESCRIPTION OF THE INVENTION

Referring now in more detail to the drawings, a shoulder sleeve support and vest is described, which employs at least an inflatable device to support a shoulder while lying supine in the open packed position (anatomical position whereby the shoulder has the least amount of stress).

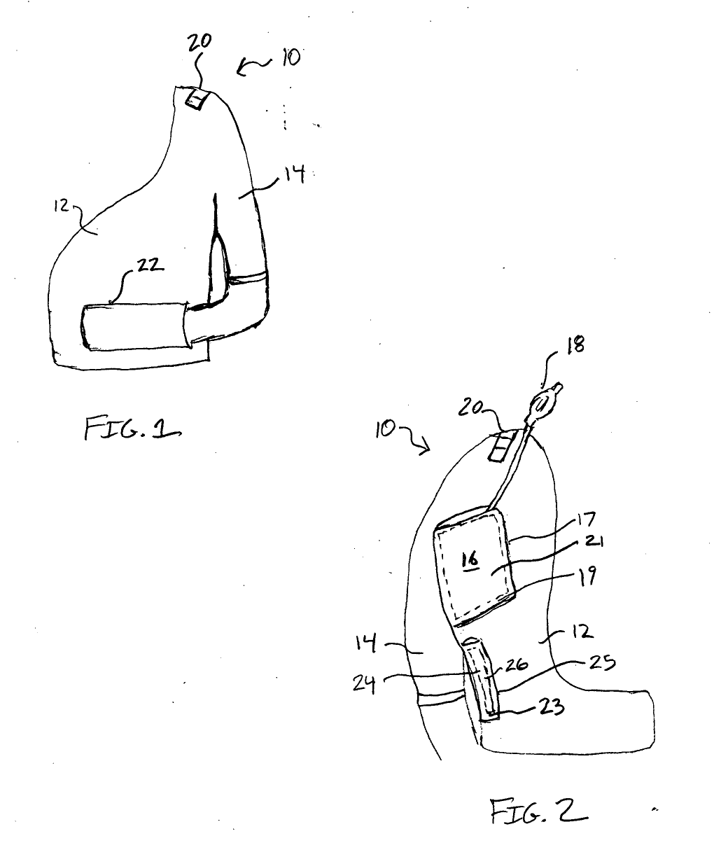

Referring to FIG. 1 and FIG. 2, there is shown a shoulder sleeve support and vest 10, including a vest section 12 oriented on the torso of a patient, and a sleeve section 14 integrated with the vest section 12, which is placed on an affected shoulder and arm portion of the patient. The shoulder sleeve and vest 10 is preferably made of a soft, aerated material, so that it is light and comfortable.

An inflatable bladder 16 is removably attached in the vicinity where the sleeve portion 14 and vest portion 12 are joined. An air pump 18 is attached to the inflatable bladder 16, and is positioned to be accessible by the patient, using their unaffected arm/hand. The patient uses the air pump 18 to inflate the inflatable bladder 16 to support the affected shoulder of the patient. The air pump 18 is preferably a blub-type hand pump, positioned so it is accessible by the patient's unaffected arm and hand. One of ordinary skill in the art would understand that other conventional air pump mechanisms may be employed, so long as they are accessible to the patient's unaffected arm/hand. Preferably the inflatable bladder 16 will only allow up to 60 degrees of shoulder flexion, as 0-60 degrees is optimal for people during the first several weeks of shoulder surgery.

The sleeve and vest 10 can also include a strap 20 attached to a front portion of the vest 12, extending to the back of the affected shoulder. Preferably, the strap 20 extends diagonally toward the back of the affected shoulder. In addition, an outer pouch 22, or “quarterback sleeve”, can be attached to the lower front portion of the vest 10 to support the forearm extending along the pouch 22, especially while the patient is lying flat.

The shoulder sleeve and vest 10 is designed to support a shoulder while lying supine in the open packed position (anatomical position which the shoulder has the least amount of stress). The inflatable device 16 will attach to the back of the sleeve 14 and vest 12 and lay on the superior (towards head)/medial (towards midline) boarder of the scapula, and support the posterior (back) aspect of the shoulder joint.

As described above, the inflatable bladder 16 is removably attached in the vicinity where the sleeve portion 14 and vest portion 12 are joined. Preferably, a first inner pouch 17 is provided to house the inflatable bladder 16. In this embodiment, an inner pouch liner 19 would have a hook and loop fastener material, that would mate with a hook and loop fastener material 21 on the exterior of the inflatable bladder 16. In such a case, a doctor could adjust the positioning of the inflatable bladder 16 within the first inner pouch 17 to suit the unique anatomy of each individual. The mating hook and loop fasteners ensure the inflatable bladder 16 will not move once positioned by the doctor.

The strap 20 is essentially a scapula strap, extending from the front of the vest 12 toward the back of the shoulder. The strap 20 may be any conventional strap apparatus, such as a hook and loop fastener strap, or a web/buckle strap. The hook and loop fastener strap is preferable, because fastening and adjusting can be accomplished with a single arm/hand operation. As with the inflatable bladder 16, the strap 20 can be initially positioned by a doctor, conforming to the unique anatomy of the patient. Thereafter, the patient can ensure the correct placement by mimicking the initial placement of the strap 20 by the doctor.

In addition to the adjustable inflatable bladder 16 and the adjustable strap 20, there is shown in FIG. 2, a third adjustable support mechanism comprising a foam material 24, and preferably the foam material 24 is memory foam. The foam material 24 will be oriented on the side of the vest 12, adjacent to the patient. This foam material 24 is designed to cradle the arm, prevent extension, and to assist with shoulder abduction (movement of arm to the side). The foam material 24 essentially helps position the humerus in the midline of the body.

Preferably, a second inner pouch 25 is provided to house the foam material 24. In this embodiment, the liner 23 of the second inner pouch 25 would have a hook and loop fastener material, that would mate with a hook and loop fastener material 26 on the foam material 24. In such a case, a doctor could adjust the positioning of the foam material 24 within the second inner pouch 25 to suit the unique anatomy of each individual. The mating hook and loop fasteners ensure the foam material 24 will not move once positioned by the doctor. Preferably the foam material 24 will only allow up to 60 degrees of shoulder abduction (movement of arm to the side), as 0-60 degrees is optimal for people during the first several weeks of shoulder surgery.

One of ordinary skill in the art would understand that the exact locations and sizes of many of the features of the present invention, including for example, the inflatable bladder 16, first inner pouch 17, strap 20, outer pouch 22, foam material 24 and second inner pouch 25, may be varied within the scope of the present invention. What matters is that the features are of sufficient size and placement to accommodate placement by a doctor in a desired position, based on the individual anatomy of the patient.

The shoulder support of the present invention is designed to maintain the typical anatomical position of the humeral head in the glenoid (center the ball in the socket), inhibit superior elevation of the scapula, and/or position the humerus in the midline of the body. The shoulder support of the present invention is fully adjustable, allowing health care practitioners to adjust the brace to meet the individual needs and anatomy of each patient.

This invention therefore provides a inflatable shoulder sleeve and vest that will fully support the arm for comfort and protect the injured extremity while laying flat on the unaffected side. This brace is designed to prevent movement of the arm throughout the night for a safe and confortable sleep.

What is claimed is:

1. A shoulder support sleeve and vest, comprising:
a vest section comprised of a fabric material and oriented on the torso of a patient;
a sleeve section comprised of a fabric material, and integrated with the vest section, for placement on an affected shoulder and arm portion of the patient;
an inflatable bladder attached to a back of the sleeve section and the vest section; and
an air pump attached to the inflatable bladder, and accessible by the patient's unaffected arm and hand,
whereby the patient uses the air pump to inflate the inflatable bladder device to a desired size such that the inflatable bladder supports the affected shoulder of the patient.

2. The shoulder support sleeve and vest of claim 1, wherein the inflatable bladder is removably attached to the sleeve section and the vest section.

3. The shoulder support sleeve and vest of claim 2, wherein the inflatable bladder is removably attached via hook and loop fasteners affixed to the inflatable bladder, sleeve section and vest section.

4. The shoulder support sleeve and vest of claim 1, further comprising a foam material placed on a side of the vest adjacent to the patient.

5. The shoulder support sleeve and vest of claim 4, wherein the foam material is removably attached to the side of the vest section.

6. The shoulder support sleeve and vest of claim 4, further comprising a strap attached to a front portion of the vest, and extending toward the back of the affected shoulder.

7. The shoulder support sleeve and vest of claim 6, wherein the strap extends diagonally toward the back of the affected shoulder.

8. The shoulder support sleeve and vest of claim 6, further comprising an outer pouch attached to a lower portion of the vest.

9. The shoulder support sleeve and vest of claim 3, further comprising a first inner pouch for receiving the inflatable bladder, the first inner pouch having an inner liner comprising a hook and loop fastener material, which mates with a hook and loop fastener material affixed to the inflatable bladder.

10. The shoulder support sleeve and vest of claim 5, further comprising a second inner pouch for receiving the foam material, the second inner pouch having an inner liner comprising a hook and loop fastener material, which mates with a hook and loop fastener material affixed to the foam material.

11. The shoulder support sleeve and vest of claim 1, wherein the fabric material is a light, aerated fabric.

12. The shoulder support sleeve and vest of claim 4, wherein the foam material is a memory foam.

13. The shoulder support sleeve and vest of claim 6, wherein the strap is a hook and loop fastener apparatus.

14. The shoulder support sleeve and vest of claim 1, wherein the desired size of the inflatable balder is limited to provide up to 60 degrees of shoulder flexion.

15. A method of supporting a shoulder while lying supine, the method comprising:
providing a vest section comprised of a fabric material and oriented on the torso of a patient;
providing a sleeve section comprised of a fabric material, and integrated with the vest section, for placement on an affected shoulder and arm portion of the patient;
providing an inflatable bladder attached to a back of the sleeve section and vest section;
providing an air pump attached to the inflatable bladder, and accessible by the patient's unaffected arm and hand; and
inflating the inflatable bladder device to a desired size to enable the inflatable bladder to support the affected shoulder of the patient.

16. The shoulder support sleeve and vest of claim 15, further comprising a step of removably attaching the inflatable bladder to the sleeve section and the vest section.

17. The method of claim 16, further comprising a step of limiting the size of the inflatable balder such that only 60 degrees of shoulder flexion can be achieved.
